(19) United States
(12) Patent Application Publication
(10) Pub. No.: US 2018/0164275 A1
(43) Pub. Date: Jun. 14, 2018

(54) METHOD OF DETERMINING THE MOLECULAR WEIGHT DISTRIBUTION OF GLATIRAMER ACETATE USING MULTI-ANGLE LASER LIGHT SCATTERING (MALLS)

(71) Applicants: Avraham Arthur KOMILOSH, Binyamina (IL); Dalia PINKERT, Kfar-Sava (IL)

(72) Inventors: Avraham Arthur KOMILOSH, Binyamina (IL); Dalia PINKERT, Kfar-Sava (IL)

(73) Assignee: Teva Pharmaceutical Industries Ltd., Petach Tikva (IL)

(21) Appl. No.: 15/570,030

(22) PCT Filed: Apr. 29, 2016
(86) PCT No.: PCT/US2016/030277

§ 371 Date: Oct. 27, 2017

Related U.S. Application Data
(60) Provisional application No. 62/155,236, filed on Apr. 30, 2015.

(51) Int. Cl. G01N 33/44; C08G 69/10; G01N 21/47; G01N 30/89; G01N 30/06; C08G 69/46

(52) U.S. Cl. CPC G01N 33/442; C08G 69/10; G01N 21/47; G01N 2021/4711; G01N 30/06; C08G 69/46; G01N 30/89

(57) ABSTRACT

The present invention provides a process for characterizing a glatiramer acetate, related drug substance (GARDS) or a glatiramer acetate related drug product (GARDP) comprising separating a batch of a GARDS or GARDP according to hydrophobicity and determining the molar mass of the separated material, thereby characterizing the GARDS or GARDP by molar mass as a function of hydrophobicity.

This application claims priority of U.S. Provisional Application No. 62/155,236, filed Apr. 30, 2016, the content of which is hereby incorporated by reference.

Throughout this application, various publications are referenced, including referenced by Arabic numerals. Full citations for publications referenced in Arabic numerals may be found listed at the end of the specification immediately preceding the claims. The disclosures of all referenced publications in their entireties are hereby incorporated by reference into this application in order to store fully describe the state of the art to which this invention pertains.

### BACKGROUND OF THE INVENTION

Multiple sclerosis (MS) is a chronic, debilitating autoimmune disease of the central nervous system (CNS) with either relapsing-remitting (RR) or progressive course leading to neurologic deterioration and disability. At time of initial diagnosis, RRMS is the most common form of the disease (1) which is characterized by unpredictable acute episodes of neurological dysfunction (relapses), followed by variable recovery and periods of clinical stability. The vast majority of RRMS patients eventually develop secondary progressive (SP) disease with or without superimposed relapses. Around 15% of patients develop a sustained deterioration of their neurological function from the beginning; this form is called primary progressive (PP) MS. Patients who have experienced a single clinical event (Clinically Isolated Syndrome or “CIS”) and who show lesion dissemination on subsequent magnetic resonance imaging (MRI) scans according to McDonald's criteria, are also considered as having relapsing MS (2).

With a prevalence that varies considerably around the world, MS is the most common cause of chronic neurological disability in young adults (3, 4). Anderson et al. estimated that there were about 350,000 physician-diagnosed patients with MS in the United States in 1990 (approx. 140 per 100,000 population) (5). It is estimated that about 2.5 million individuals are affected worldwide (6). In general, there has been a trend toward an increasing prevalence and incidence of MS worldwide, but the reasons for this trend are not fully understood (5).

Current therapeutic approaches consist of i) symptomatic treatment ii) treatment of acute relapses with corticosteroids and iii) treatment aimed to modify the course of the disease. Currently approved therapies target the inflammatory processes of the disease. Most of them are considered to act as immunomodulators but their mechanisms of action have not been completely elucidated. Immunosuppressants or cytotoxic agents are also used in some patients after failure of conventional therapies. Several medications have been approved and clinically ascertained as efficacious for the treatment of RR-MS; including BETASERON®, AVONEX® and REBIF®, which are derivatives of the cytokine interferon beta (IFNB), whose mechanism of action in MS is generally attributed to its immunomodulatory effects, antagonizing pro-inflammatory reactions and inducing suppressor cells. Other approved drugs for the treatment of MS include Mitoxantrone and Natalizumab (7).

Copaxone® (Teva Pharmaceutical Industries Ltd.) is indicated for the treatment of patients with relapsing forms of multiple sclerosis (8). Copaxone® is a clear, colorless to slightly yellow, sterile, nonpyrogenic solution for subcutaneous injection (8). Each 1 mL of Copaxone® solution contains 20 mg or 40 mg of the active ingredient, glatiramer acetate (GA), the inactive ingredient, 40 mg of mannitol (8).

GA, the active ingredient of Copaxone®, consists of the acetate salts of synthetic polypeptides, containing four naturally occurring amino acids: L-glutamic acid, L-alanine, L-tyrosine, and L-lysine with an average solar fraction of 0.141, 0.427, 0.095, and 0.338, respectively (8). Glatiramer acetate is identified by specific antibodies (8).

GA elicits anti-inflammatory as well as neuroprotective effects in various animal models of chronic inflammatory and neurodegenerative diseases (9-13) and has been shown to be safe and effective in reducing relapses and delaying neurologic disability in MS patients following long-term treatment (14).

The mechanisms underlying GA therapeutic activity are not fully elucidated, but GA activity on immune cells has been well demonstrated. GA appears to act as an altered peptide ligand (APL) of encephalitogenic epitopes within myelin basic protein (MBP) (15) and demonstrates cross-reactivity with MBP at the humoral and cellular levels (16-22). The unique antigenic sequences of the GA polypeptide mixture compete with myelin antigens for binding to MHC class II molecules on antigen presenting cells (APCs) and presentation to the T cell receptor (TCR), resulting in the induction of energy or deletion of autoreactive MBP-reactive T cells and proliferation of GA-reactive T cells. At initiation of Copaxone® treatment, GA-reactive CD4+ T-cell lines from MS patients secrete both pro-inflammatory T helper type 1 (Th1) and anti-inflammatory Th2 cytokines (20, 23), but continued exposure to Copaxone® induces a shift, in GA-reactive T cells toward the Th2 phenotype (20, 22, 24-27). In MS patients treated with daily subcutaneous Copaxone 20 mg/ml, anti-GA antibody peaked at 3 months after initiation of treatment, decreasing at 6 months and remaining low, and IgG1 antibody levels were 2-3 fold higher than those of IgG2 (28).

Copaxone® also increases the number and suppressive capacity of CD4+CD25+FOXP3+ regulatory T cells, which are functionally impaired in MS patients (29-31). Furthermore, treatment leads to antigen-nonspecific modulation of APC function. Copaxone® treatment promotes development of anti-inflammatory type II monocytes characterized by an increase in interleukin (IL)-10 and transforming growth factor-beta (TGF-β) and decreased production of IL-12 and tumor necrosis factor (TNF) (32).

### SUMMARY OF THE INVENTION

The present invention provides a process for characterizing a glatiramer acetate related drug substance (GARDS) or a glatiramer acetate related drug product (GARDP)comprising separating a batch of a GARDS or GARDP according to hydrophobicity and determining the molar mass of the separated material, thereby characterizing the GARDS or GARDP by molar mass as a function of hydrophobicity.

The present invention also provides a process for discriminating between two or more GARDSs or GARDPs comprising:
(I) characterizing two or more GARDSs or GARDPs according to the process of the present invention to obtain a profile of molar mass as a function of hydrophobicity for each of the two or more GARDS or GARDP; and
(II) comparing each of the profiles obtained in step (I) to each other,
   thereby discriminating between the GARDSs or GARDPs.

The present invention also provides a process for producing a drug product comprising a GARDS, which involves an array of testing, comprising including in the array of testing:
(I) characterizing a GARDS according to the process of the present invention to obtain a profile of molar mass as a function of hydrophobicity for the GARDS;
(II) characterizing glatiramer acetate drug substance (GADS) according to the same conditions used in step (I) to obtain a profile of molar mass as a function of hydrophobicity for GADS; and
(III) including the GARDS in the production of the drug product if the profile obtained in step (I) is substantially equivalent to the profile obtained in step (II).

The present invention also provides a process for releasing a drug product comprising a GARDS, which involves an array of testing, comprising including in the array of testing:
(I) characterizing a GARDS according to the process of the present invention to obtain a profile of molar mass as a function of hydrophobicity for the GARDS;
(II) characterizing glatiramer acetate drug substance (GADS) according to the same conditions used in step (I) to obtain a profile of molar mass as a function of hydrophobicity for GADS; and
(III) releasing the drug product if the profile obtained in step (I) is substantially equivalent to the profile obtained in step (II).

The present invention also provides a process for identifying GARDS or GARDP that has suboptimal activity comprising:

(I) characterizing a GARDS according to the process the present invention to obtain a profile of molar mass as a function of hydrophobicity for the GARDS;

(II) characterizing glatiramer acetate drug substance (GADS) according to the same conditions used in step (I) to obtain a profile of molar mass as a function of hydrophobicity for GADS; and

(III) identifying the GARDS or GARDP as having a suboptimal activity if the profile obtained in step (I) is not substantially equivalent to the profile obtained in step (II).

### BRIEF DESCRIPTION OF THE DRAWINGS

FIG. 1. Eighteen photodetectors spaced in a multi-angle geometry.

FIG. 2. Debye plot.

FIG. 3. UV absorbance and molar mass profiles of a representative Copaxone® batch as a function of retention time.

FIG. 4. Overlay of molar mass as a function of retention time profiles of 5 Copaxone® batches.

FIG. 5A. Overlay of molar mass as a function of retention time profiles of 5 Copaxone® batches and Polimunol batch A.

FIG. 5A. Overlay of molar mass as a function of retention time profiles of 5 Copaxone® batches and Polimunol batch B.

FIG. 6A. Overlay of molar mass as a function of retention time profiles of 5 Copaxone® batches and Glatimer batch A.

FIG. 6B. Overlay of molar mass as a function of retention time profiles of 5 Copaxone® batches and Glatimer batch B.

FIG. 7A. Overlay of molar mass as a function of retention time profiles of 5 Copaxone® batches and Escadra batch A.

FIG. 7B. Overlay of molar mass as a function of retention time profiles of 5 Copaxone® batches and Escadra batch B.

FIG. 8. Overlay of molar mass as a function of retention time profiles of 5 Copaxone® batches and Probioglat batch A.

### DETAILED DESCRIPTION OF THE INVENTION

The present invention provides a process for characterizing a glatiramer acetate related drug substance (GARDS) or a glatiramer acetate related drug product (GARDP) comprising separating a batch of a GARDS or GARDP according to hydrophobicity and determining the molar mass of the separated material, thereby characterizing the GARDS or GARDP by molar mass as a function of hydrophobicity.

In some embodiments the process further comprising a step of producing a profile of the molar mass of the GARDS or GARDP.

In some embodiments separating is performed by eluting the batch of the GARDS or GARDP using chromatography with a mobile phase.

In some embodiments the chromatography is reversed-phase chromatography.

In some embodiments the reversed-phase chromatography is reversed-phase high-performance liquid chromatography.

In some embodiments the chromatography is performed with a gradient elution of the mobile phase.

In some embodiments the gradient elution is achieved by using organic solvent up to 50% by volume of the mobile phase.

In some embodiments the organic solvent is 0.1% trifluoroacetic acid in acetonitrile.

In some embodiments the batch of the GARDS or GARDP is separated into a continuous stream having varying hydrophobicity and the molar mass of at least a portion of the continuous stream is determined.

In some embodiments the batch of the GARDS or GARDP is separated into separate fractions having varying hydrophobicity and the molar mass of a separated fraction is determined.

In some embodiments the molar mass is determined using a Multi Angle Laser Light Scattering (MALLS) instrument.

In some embodiments the profile is a profile of molar mass as a function of hydrophobicity.

The present invention also provides a process for discriminating between two or more GARDSs or GARDPs comprising:
(I) characterizing two or more GARDSs or GARDPs according to the process of the present invention to obtain a profile of molar mass as a function of hydrophobicity for each of the two or more GARDS or GARDP; and
(II) comparing each of the profiles obtained in step (I) to each other,
thereby discriminating between the GARDSs or GARDPs.

In some embodiments the characterization is by chromatography, further comprising the step of identifying the GARDSs or GARDPs as not substantially equivalent if:
the peak molar mass of the GARDSs or GARDPs according to the profiles are different; or
the retention time at the peak of the profiles of the GARDSs or GARDPs are different.

The present invention also provides a process for producing a drug product comprising a GARDS, which involves an array of testing, comprising including in the array of testing:
(I) characterizing a GARDS according to the process of the present invention to obtain a profile of molar mass as a function of hydrophobicity for the GARDS;
(II) characterizing glatiramer acetate drug substance (GADS) according to the same conditions used in step (I) to obtain a profile of molar mass as a function of hydrophobicity for GADS; and
(III) including the GARDS in the production of the drug product if the profile obtained in step (I) is substantially equivalent to the profile obtained in step (II).

The present invention also provides a process for producing a drug product comprising a GARDS, which involves an array of testing, comprising including in the array of testing:
(I) characterizing a GARDS according to the process of the present invention to obtain a profile of molar mass as a function of hydrophobicity for the GARDS; and
(II) including the GARDS in the production of the drug product if the profile obtained in step (I) is substantially equivalent to the profile representing glatiramer acetate drug substance (GADS) when characterized under the same conditions as the conditions used in step (I).

The present invention also provides a process for producing a drug product comprising a GARDS, which involves an array of testing, comprising including in the array of testing:
(a) characterizing a GARDS according to the process of the present invention to obtain a profile of molar mass as a function of hydrophobicity for the GARDS; and
(b) including the GARDS in the production of the drug product if the profile has a single peak and the peak molar mass of the GARDS according to the profile is in the range of 8,000-10,000 g/mol.

In some embodiments the characterization is by chromatography, further comprising:
(I) characterizing glatiramer acetate drug substance (GADS) according to the same conditions used in step (a) to obtain a profile of molar mass as a function of hydrophobicity for GADS; and
(II) including the GARDS in the production of the drug product if the chromatography retention time at the peak molar mass of the GARDS is substantially equivalent to the chromatography retention time at the peak molar mass of the GADS.

The present invention also provides a process for releasing a drug product comprising a GARDS, which involves an array of testing, comprising including in the array of testing:
(I) characterizing a GARDS according to the process of the present invention to obtain a profile of molar mass as a function of hydrophobicity for the GARDS;
(II) characterizing glatiramer acetate drug substance (GADS) according to the same conditions used in step (I) to obtain a profile of molar mass as a function of hydrophobicity for GADS; and
(III) releasing the drug product if the profile obtained in step (I) is substantially equivalent to the profile obtained in step (II).

The present invention also provides a process for releasing a drug product comprising a GARDS, which involves an array of testing, comprising including in the array of testing:
(I) characterizing a GARDS according to the process of the present invention to obtain a profile of molar mass as a function of hydrophobicity for the GARDS; and
(II) releasing the drug product if the profile obtained in step (I) is substantially equivalent to the profile representing glatiramer acetate drug substance (GADS) when characterized under the same conditions as the conditions used in step (I).

The present invention also provides a process for releasing a drug product comprising a GARDS, which involves an array or testing, comprising including in the array of testing:
(a) characterizing a GARDS according to the process of the present invention to obtain a profile of molar mass as a function of hydrophobicity for the GARDS; and
(b) releasing the drug product if the profile has a single peak and the peak molar mass of the GARDS according to the profile is in the range of 8,000-10,000 g/mol.

In some embodiments the characterization is by chromatography, further comprising:
(I) characterizing glatiramer acetate drug substance (GADS) according to the same conditions used in step (a) to obtain a profile of molar mass as a function of hydrophobicity for GADS; and
(II) releasing the drug product if the chromatography retention time at the peak molar mass of the GARDS is substantially equivalent to the chromatography retention time at the peak molar mass of the GADS.

The present invention also provides a process for identifying GARDS or GARDP that has suboptimal activity comprising:
(I) characterizing a GARDS according to the process the present invention to obtain a profile of molar mass as a function of hydrophobicity for the GARDS;
(II) characterizing glatiramer acetate drug substance (GADS) according to the same conditions used in step (I) to obtain a profile of molar mass as a function of hydrophobicity for GADS; and
(III) identifying the GARDS or GARDP as having a suboptimal activity if the profile obtained in step (I) is not substantially equivalent to the profile obtained in step (II).

The present invention also provides a process for identifying GARDS or GARDP that has suboptimal activity comprising:
(I) characterizing a GARDS according to the process of the present invention to obtain a profile of molar mass as a function of hydrophobicity for the GARDS;
(II) identifying the GARDS or GARDP as having a suboptimal activity if the profile obtained in step (I) is not substantially equivalent to the profile representing glatiramer acetate drug substance (GADS) when characterized under the same conditions as the conditions used in step (I).

The present invention also provides a process for identifying GARDS or GARDP that has suboptimal activity comprising:
(a) characterizing a GARDS according to the process of the present invention to obtain a profile of molar mass as a function of hydrophobicity for the GARDS; and
(b) identifying the GARDS or GARDP as having a suboptimal activity if the profile has more than one peak or the peak molar mass of the GARDS according to the profile is not in the range of 8,000-10,000 g/mol.

In some embodiments the characterization is by chromatography, further comprising:
(I) characterizing glatiramer acetate drug substance (GADS) according to the same conditions used in step (a) to obtain a profile of molar mass as a function of hydrophobicity for GADS; and
(II) identifying the GARDS or GARDP as having a suboptimal activity if the chromatography retention time at the peak molar mass of the GARDS is not substantially equivalent to the chromatography retention time at the peak molar mass of the GADS.

In some embodiments, the difference between the peak molar masses of the GARDSs or GARDPs is greater than 10% of the highest peak molar mass value between the GARDSs or GARDPs.

In some embodiments, the difference between the peak molar masses of the GARDSs or GARDPs is greater than 5% of the highest peak molar mass value between the GARDSs or GARDPs.

In some embodiments, the difference between the peak molar masses of the GARDSs or GARDPs is greater than 1% of the highest peak molar mass value between the GARDSs or GARDPs.

In some embodiments, the difference between the retention time at the peak of the profiles of the GARDSs or GARDPs is greater than 10% of the latest retention time at the peak of the profiles between the GARDS or GARDP.

In some embodiments, the difference between the retention time at the peak of the profiles of the GARDSs or GARDPs is greater than 5% of the latest retention time at the peak of the profiles between the GARDS or GARDP.

In some embodiments, the difference between the retention time at the peak of the profiles of the GARDSs or GARDPs is greater than 1% of the latest retention time at the peak of the profiles between the GARDS or GARDP.

There are multiple ways of separating polypeptide mixtures with chromatography and determining the molar mass of the separated polypeptide mixtures with MALLS. For example, polypeptide mixtures can be eluted based on hydrophobicity in a continuous flow using high performance liquid chromatography and the molar mass of the flow can be determined continuously with MALLS. Polypeptide mixtures can also be eluted into separate fractions using various types of reversed phase chromatography and the molar mass of the separate fractions can be determined intermittently. Determination of molar mass of separate fractions can be achieved by many different means including but not limited to using MALLS as well as molecular weight markers as disclosed in U.S. Pat. Nos. 6,800,287, 7,074,580, 7,163,802, 7,615,359 and 8,399,211, the disclosures of which are hereby incorporated by reference in their entireties.

Each embodiment disclosed herein is contemplated as being applicable to each of the other disclosed embodiments. Thus, all combinations of the various elements described herein are within the scope of the invention.

### Definitions

As used herein, “glatiramer acetate” is a complex mixture of the acetate salts of synthetic polypeptides, containing four naturally occurring amino acids: L-glutamic acid, L-alanine, L-tyrosine, and L-lysine. The peak average molecular weight of glatiramer acetate is between 5,000 and 9,000 daltons. Chemically, glatiramer acetate is designated L-glutamic acid polymer with L-alanine, L-lysine and L-tyrosine, acetate (salt). Its structural formula is:
(Glu, Ala, Lys, Tyr)x.X CH3COOH
C₅H₉NO₄.C₃H₇NO₂.C₆H₁₄N₂O₂.C₉H₁₁NO₃)x.XC₂H₄O₂
CAS-147245-92-10 (8).

As used herein, the term “glatiramer acetate related drug substance” (GARDS) is intended to include polypeptides with a predetermined sequence as well as mixtures of polypeptides assembled from the four amino acids glutamic acid (E), alanine (A), lysine (K), and tyrosine (Y); from any three of the amino acids Y, E, A and K, i.e. YAK, YEK, YEA or EAK; or from three of the amino acids Y, E, A and K and a fourth amino acid. Examples of glatiramer acetate related polypeptides are disclosed in U.S. Pat. Nos. 6,514,938 A1, 7,279,172 B2, 7,560,100 and 7,655,221 B2 and U.S. Patent Application Publication No. US 2000-0131170 A1, the disclosures of which are hereby incorporated by reference in their entireties. Glatiramer acetate related substances include glatiramoids.

As used herein, a “glatiramer acetate related drug product” (GARDP) contains a glatiramer acetate related drug substance.

As used herein a “glatiramoid” is a complex mixture of synthetic proteins and polypeptides of varying sizes assembled from four naturally occurring amino acids: L-glutamic acid, L-alanine, L-lysine, and L-tyrosine. Examples of glatiramoids include glatiramer acetate drug substance (e.g. the active of Copaxone®) as well as other polypeptides, e.g. GA-Natco.

As used, herein, a “glatiramer acetate drug substance” (GADS) is glatiramer acetate produced by Teva Pharmaceutical Industries, Ltd. and is the active ingredient in a glatiramer acetate drug product.

As used herein, a “glatiramer acetate drug product” (GADP) contains a glatiramer acetate drug substance produced by Teva Pharmaceutical Industries, Ltd.

As used herein, a “glatiramer acetate drug substance or drug product” is a glatiramer acetate drug substance or a glatiramer acetate drug product.

In certain embodiments of the invention, “molar mass” or “absolute molecular weight” may be calculated as a function of sample concentration and the scattered light ratio as seen in the following equation:

$$M W ≅ \frac{R ( θ )}{K \times C}$$

### Where:

MW is the absolute molecular weight;
R(θ) is the scattering ratio that would be obtained at angle of zero;
K is an optical constant ˜(dn/dc)²; and
C is the polymer concentration in solution.

As used herein, the term “retention time” or “elution time” is the time required for protein or polypeptide to elute from a column.

As used herein, “release” of a drug product refers to making the product available to consumers.

As used herein, an “array of testing” for a glatiramer acetate related drug substance or drug product includes, but is not limited to, any analytical method test such as in vitro tests or molecular weight tests, biological assays such as the ex vivo tests and clinical efficacy tests which characterize the GARDS or GARDP, or clinical trials. Examples of testing for a glatiramer acetate related drug substance or drug product are disclosed in U.S. Patent Application Publication Nos. US 2012-0309671 and US 2011-0230413, and in PCT International Application Publication Nos. WO 2000/018794, WO 2012/051106, WO 2013/055683, WO 2014/058976, the disclosures of which are hereby incorporated by reference in their entireties.

As used herein, “characterization” or “characterizing” is understood to include obtaining information which was produced in the same location or country, or a different location or country from where any remaining steps of the method are performed.

As used herein, “2D profile” is a two-dimensional profile, for example a profile of the molar mass as a function of hydrophobicity for GARDS or GARDP.

As used herein, “a profile of molar mass as a function of hydrophobicity” includes a profile of molar mass as a function of hydrophobicity, of retention time, or any other parameter as long as the retention time or the other parameter correlates with hydrophobicity of the material being characterized.

As used herein, the term “substantially equivalent” when used in the context of a profile of molar mass as a function of hydrophobicity means that each point in a profile is within 10%, preferably 5%, most preferably 1% of each corresponding point of a profile obtained under the same conditions for a reference material. In a specific example the term “substantially equivalent” refers to a point of molar mass as a function of hydrophobicity in a profile which is within 10%, preferably 5%, most preferably 1% of a corresponding molar mass point as a function of hydrophobicity of a profile obtained under the same conditions for a reference material.

It is understood that where a parameter range is provided, all integers within that range, tenths thereof, and hundredths thereof, are also provided by the invention. For example, “0.2-5 mg” is a disclosure of 0.2 mg, 0.21 mg, 0.22 mg, 0.23 mg etc. up to 0.3 mg, 0.31 mg, 0.32 mg, 0.33 mg etc. up to 0.4 mg, 0.5 mg, 0.6 mg etc. up to 5.0 mg.

As used herein, determination of the molar mass of peptides in solution using a Multi Angle Laser Light Scattering (MALLS) instrument are known in the art. Examples are disclosed in U.S. Pat. Nos. 8,760,652 and 5,269,937, the disclosures of which are hereby incorporated by reference in their entireties.

As used herein, processes of producing a glatiramer acetate related drug substance or drug product are known in the art. Examples of such manufacturing processes are disclosed in U.S. Pat. No. 5,800,808, and in PCT International Application Publication Nos. WO 2005/032553, WO 2005/032395, WO 1999/22402, the disclosures of which are hereby incorporated by reference in their entireties.

Each embodiment disclosed herein is contemplated as being applicable to each of the other disclosed embodiments. Thus, all combinations of the various elements described herein are within the scope of the invention.

This invention will be better understood by reference to the Experimental Details which follow, but those skilled in the art will readily appreciate that the specific experiments detailed are only illustrative of the invention as described more fully in the claims which follow thereafter.

### EXPERIMENTAL DETAILS

### Example 1

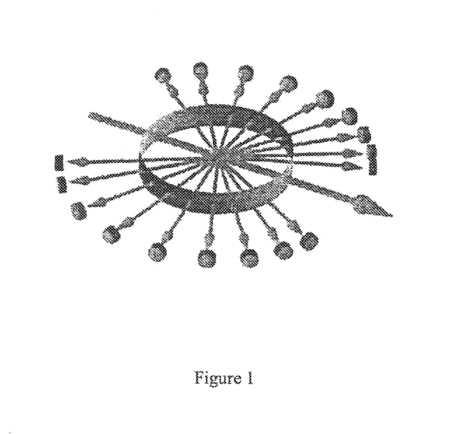

Multi Angle Laser Light (MALLS) scattering is a technique for determination of the absolute molar mass of particles in solution by detecting how they scatter light. The intensity of the scattered light is measured as a function of the scattering light angle. The DAWN HELEOS II® (Wyatt Technology) instrument can measure molar masses from hundreds to millions of Daltons. It comprises eighteen discrete photodetectors that are spaced around the cell (FIG. 1), enabling simultaneous measurement over a broad range of scattering angles.

Unlike Copaxone® identification method for Molecular Weight Distribution that uses molecular markers for molecular weight calculations, MALLS does not require external calibration standards to determine molecular weight. The MALLS detector is coupled downstream to an HPLC system where the molecular weight results are purely dependent on the light scattering signal (laser) and concentration (UV).

Typically, the MALLS detector is coupled to a Size Exclusion High Performance Liquid Chromatography (SEC-HPLC) system, where isocratic elution is applied in order to measure the absolute molar mass of samples that were separated according to size.

Molar mass is a function of sample concentration and the scattered light ratio as seen in the following equation:

$$M W ≅ \frac{R ( θ )}{K \times C}$$

### Where:

MW is the absolute molecular weight;
R(θ) is the scattering ratio that would be obtained at angle of zero;
K is an optical constant ˜(dn/dc)²; and
C is the polymer concentration in solution.

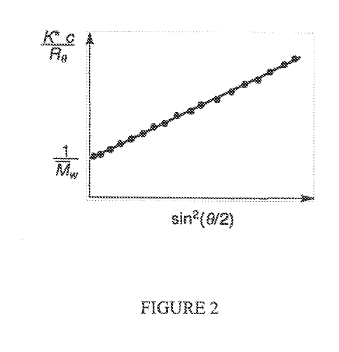

The molar mass is calculated using Debye plot, which extrapolate the scattered light intensity of the MALLS detectors at various angles to the angle of zero (FIG. 2), in light of the fact that it cannot be measured directly due to the interference of the excitating laser beam.

The purpose of the study was to combine MALLS and HPLC in a two-dimensional (2D) chromatographic technique to characterize the complex polypeptide mixtures of Copaxone® and glatiramoids other than Copaxone® based on molar mass as a function of hydrophobicity. In order to achieve the 2D separation methodology, (1) reversed-phase (“RP”) column and gradient elution were applied using an HPLC system to achieve separation based on hydrophobicity, and (2) MALLS detector to achieve separation based on molar mass.

The chromatographic conditions were based on using reverse phase column (for example: PUROSHER STAR RP-8e, 5 μm, 150×4.6 mm column) and UV detection. Elution was applied using gradient, (for example: starting from 100% of 0.1% trifluoroacetic acid (TFA) in water up to 50% of 0.1% TFA in acetonitrile (ACN) over 60 minutes).

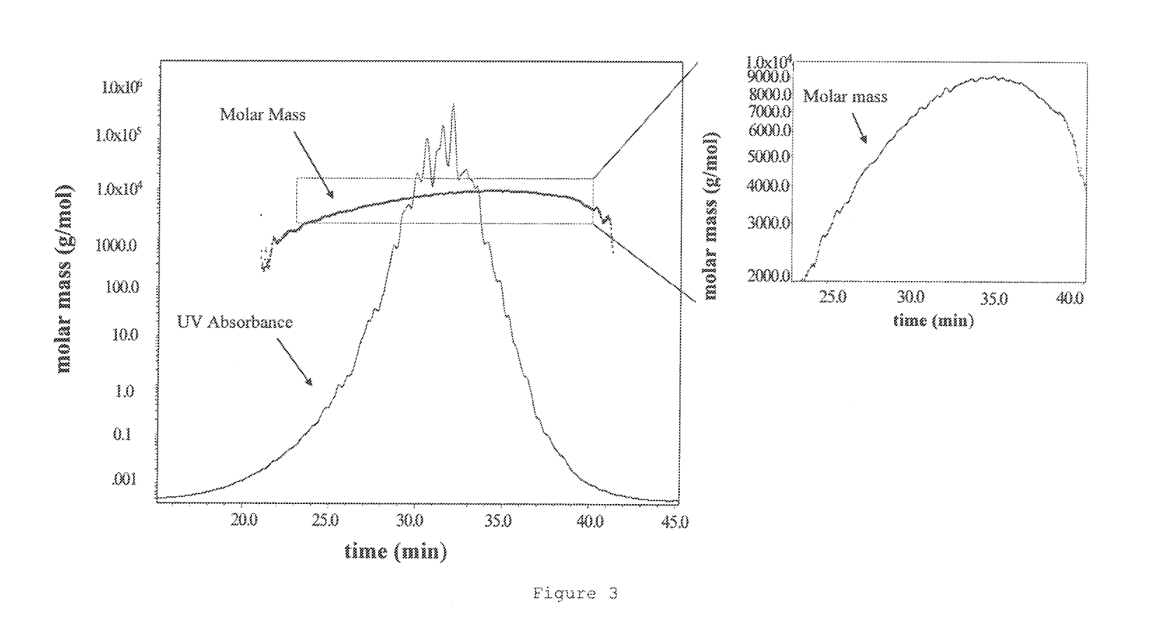

FIG. 3 presents the combined picture of the molar mass distribution profile overlaid upon the UV chromatogram of a representative Copaxone® batch and a zoomed section of the molar mass profile as a function of elution/retention time. As expected, the polypeptide mixture appears as a broad peak on the UV chromatogram, where the hydrophilic population elutes early and the hydrophobic population elutes at later retention time.

Similar molar weight profiles measured in concomitance with resolution of polypeptides on the RP column would indicate similarity of composition with regards to molar weight versus hydrophobicity, whereas differences in MALLS profiles would suggest that polypeptides with about the same hydrophobicity are characterized by different molar mass.

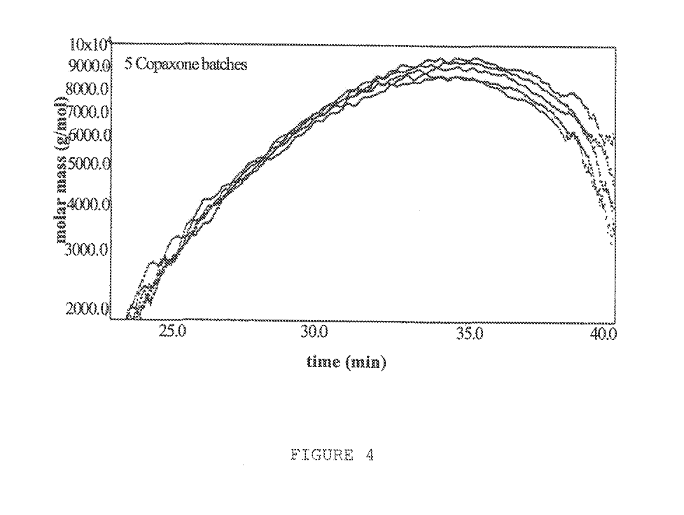

Five batches of Copaxone® 20 mg/mL analyzed on separate occasions, demonstrated consistent and repeatable results. A stack overlay (zoomed in) of the five Copaxone® batches is presented in FIG. 4.

As can be seen in FIG. 4, the five Copaxone® batches present good batch to batch repeatability. The molar mass profile reveals that the molecular weight of the hydrophilic population starts at about 2000 Daltons (in average). A maximum molecular weight of about 9500 Daltons was obtained at about 35 min (⅔ of peak width) and back down to about 5000 Daltons at 40 min where most hydrophobic peptide population was eluted. As it seems from the profile, the molecular weight of peptides comprising the complex mixture of Copaxone® is not evenly distributed along the hydrophobicity range. The latter results indicate that the methodology is truly representing 2D characterization of Copaxone®.

Seven batches of glatiramoids other than Copaxone® were analyzed in comparison to Copaxone® batches. Two batches of Polimunol by Bago (Argentina), two batches of Glatimer by Natco (India), two batches of Escadra by Raffo (Argentina) and one batch of Probioglat by Probiomed (Mexico), all products are marketed drugs in their country of origin.

### Polimunol (Bago)

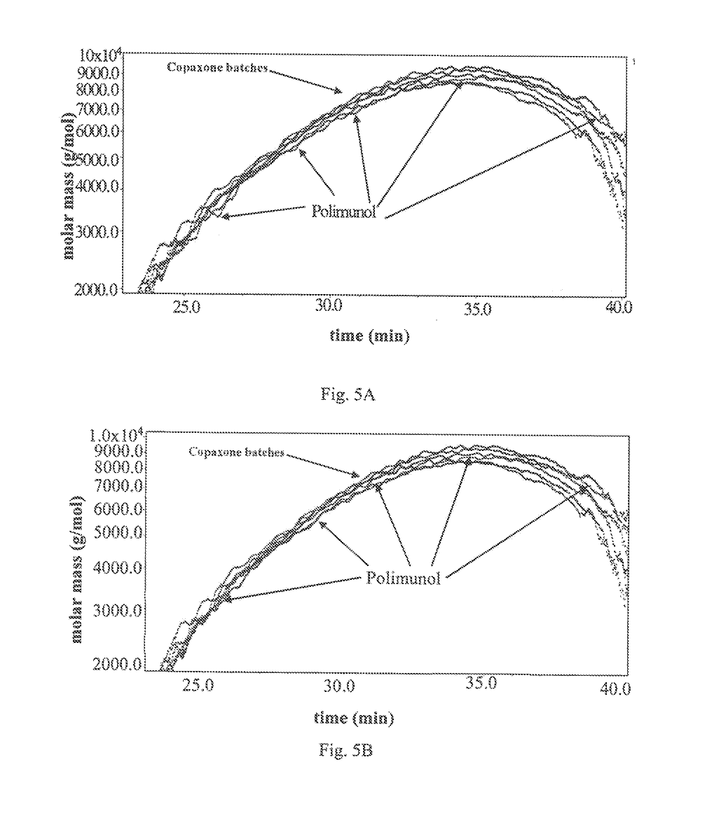

The molar mass profiles of the two tested Polimunol batches appear to be within the range of Copaxone® batches (FIG. 5A and FIG. 5B). Therefore, with regards to this method, the tested Polimunol batches seem to be comparable to Copaxone®.

### Glatimer (Natco)

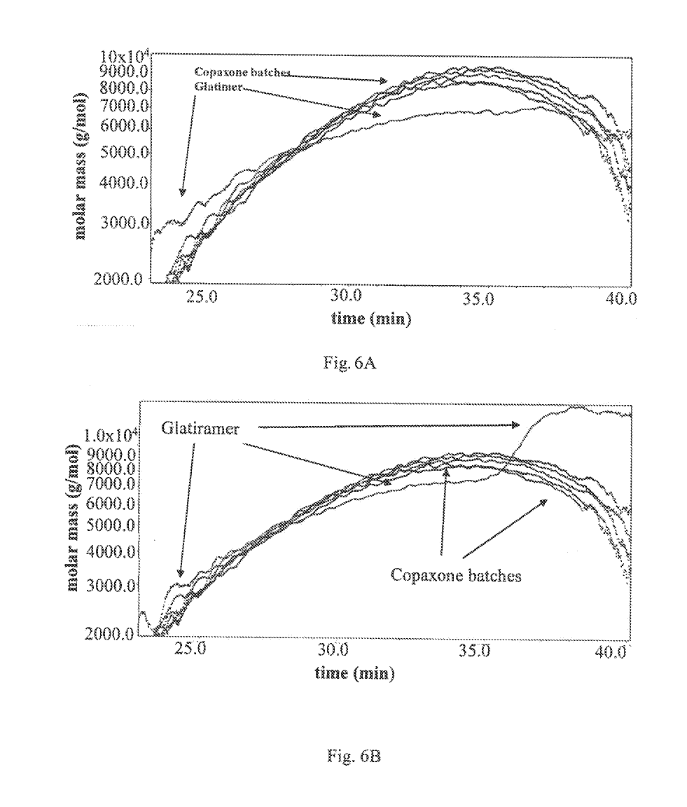

In the case of Glatimer batches, it can be observed (FIG. 6A and FIG. 6B) that both samples have different molar mass distribution profiles in comparison to Copaxone® representative batches. The Natco batches are also different from one another. Glatimer batch A (FIG. 6A) has different molar masses along the profile: a higher molar mass is observed for the hydrophilic polypeptides, at retention time of about 23-27 min and a lower molar mass of the more hydrophobic polypeptides at 29-39 min in comparison to Copaxone®. In the case of Glatimer batch B (FIG. 6B) it seems to differ from Copaxone® in the middle and in the hydrophobic parts: a lower molar mass is observed at middle part of the profile and an additional significant difference is observed at the hydrophobic part where the molar mass of the eluting polypeptides is extremely higher than that of Copaxone®. These results indicate that these batches have different composition of polypeptide mixture in comparison to Copaxone®.

### Escadra (Raffo)

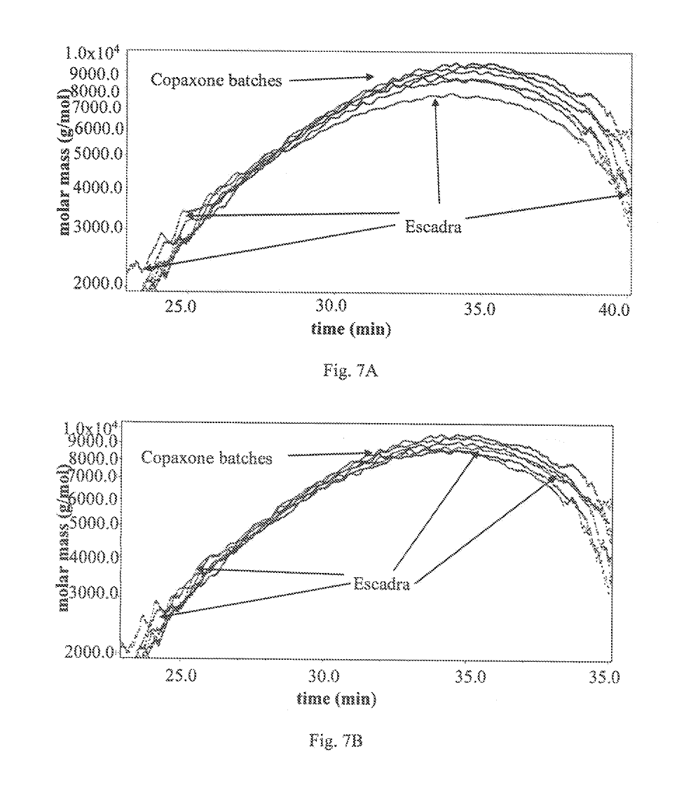

In the case of Escadra batches, it can be observed (FIG. 7A and FIG. 7B) that, with regards to this method, batch B has similar molar mass profile in comparison to Copaxone® representative batches (FIG. 7B), whereas, in the case of the batch A a lower molar mass is observed at retention time of about 29-39 min, indicating lower mass of the more hydrophobic polypeptides, in comparison to Copaxone® (FIG. 7A). These results indicate that this batch has different composition of polypeptide mixture in comparison to Copaxone®, and the batches differ one from another.

### Probioglat (Probiomed)

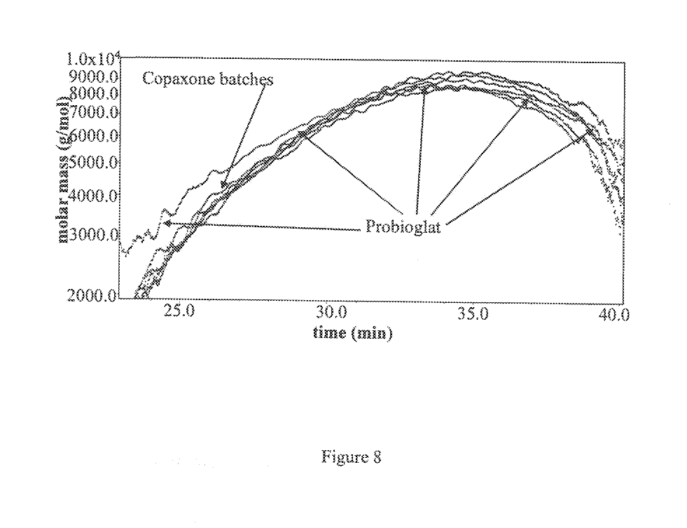

Probioglat sample seems to differ from Copaxone® mostly at the left region of the molar mass profile (FIG. 8). A higher molar mass is observed for the hydrophilic polypeptides population (at retention time of about 23-30 min) in comparison to Copaxone®, indicating, again, different composition of polypeptide mixture in comparison to Copaxone®.

### Conclusions

Analysis of 5 Copaxone® batches showed good batch to batch repeatability. The 2D chromatographic technique that characterizes polypeptide mixtures based on molar mass as a function of hydrophobicity seems to be capable of characterizing Copaxone® and discriminating it from glatiramoids other than Copaxone®.

The results of most of the glatiramoids other than Copaxone® show differences within their molar mass profiles (as a function of hydrophobicity) in comparison to Copaxone®, which reflects significant differences in the polypeptide chain compositions. These results indicate meaningful difference between Copaxone® and glatiramoids other than Copaxone®.

Discussion

A mixture can be separated according to molar mass, hydrophobicity, non-covalent interaction, ionic interaction or chirality. Separation and analysis based on a single parameter may or may not be sufficient for characterizing complex polypeptide mixtures.

The disclosed method of utilizing multi-dimensional separation and characterization of complex polypeptide mixtures offers more information about the mixture that would not have been observed without the extra dimension of separation.

The exemplified method combines MALLS and RP HPLC to achieve two dimensional separation and characterization of Copaxone® and other GARDS or GARDP based on molar mass as a function of hydrophobicity. In order to achieve the two dimensional separation methodology, (1) RP column and gradient elution were applied using an HPLC system to achieve separation based on hydrophobicity, and (2) MALLS detection was applied to achieve separation based on molar mass.

As shown in Example 1 above, the results of the disclosed method when applied to GARDS or GARDP samples other than Copaxone® show differences within their molar mass profiles as a function of hydrophobicity in comparison to Copaxone®, which reflects significant differences in the polypeptide chain compositions.

### REFERENCES

1. Noseworthy J H, Lucchinetti C, Rodriguez M, Weinshenker B G. Multiple sclerosis, N Engl J Med 2000; 343:938-52.
2. Guideline on clinical investigation of medicinal products for the treatment of multiple sclerosis EMEA, London 16 Sep. 2006.
3. Bjartmar C, Fox R J. Pathological mechanisms and disease progression of multiple sclerosis: therapeutic implications. Drugs of Today 2002; 38:17-29.
4. Fleming J O. Diagnosis and management of multiple sclerosis. 1st ed. New York: Professional communications, Inc., 2002.
5. Anderson D W, Ellenberg J H, Leventhal C M et al, Revised estimate of the prevalence of multiple sclerosis in the United States. Ann Neurol 1992; 31:333-36.
6. Compston A, Lassmann H, McDonald I. The story of multiple sclerosis. In: Compston A, Confavreux C, Lassman H, Mcdonald I, Miller D, Noseworthy J H, Smith K, Wekerle H, editors. McAlpine's Multiple Sclerosis. London: Churchill Livingstone; 2006. p, 3-68.
7. Revel M., Pharmacol. Ther., 100 (1):49-62 (2003).
8. Copaxone, Food and Drug Administration Approved Labeling (Reference ID: 3443331) [online], TEVA Pharmaceutical Industries Ltd., 2014 [retrieved on Dec. 24, 2014], Retrieved from the Internet: <URL: www.accessdata.fda.gov/drugsatfda_docs/label/2014/020622s0891b 1.pdf>.
9Varkony H, et al. (2009) The glatiramoid class of immunomodulator drugs. Expert Opin Pharmacother 10:657-668.
10. Filippi M, et al. (2001) Glatiramer acetate reduces the proportion of new MS lesions evolving into “black holes”. Neurology 57:731-733.
11. Kipnis J, Schwartz M (2002) Dual action of glatiramer acetate (Cop-1) in the treatment of CHS autoimmune and neurodegenerative disorders. TRENDS in Molecular Medicine 8:319-323.
12. Teitelbaum D, Aharoni R, Arnon R, Sela M (1988) Specific inhibition of the T-cell response to myelin basic protein by the synthetic copolymer Cop 1. Proc Natl Acad Sci USA 85:9724-9728.
13. Putheti P, Soderstrom M, Link H, Huang Y M (2003) Effect of glatiramer acetate (Copaxone®) on CD4+CD25high T regulatory cells and their IL-10 production in multiple sclerosis. J Neuroimmunol 144:125-131.
14. Stadelmann C, et al. (2002) BDNF and gp145trkB in multiple sclerosis brain lesions: neuroprotective interactions between immune and neuronal cells? Brain 125:75-85.
15. Ford C, et al. (2010) Continuous long-term immunomodulatory therapy in relapsing multiple sclerosis: results from the 15-year analysis of the US prospective open-label study of glatiramer acetate. Mult Sclsr 16:342-350.
16. Aharoni R, Teitelbaum D, Arnon R, Sela M (1999) Copolymer 1 acts against the immunodominant epitope 82-100 of myelin basic protein by T cell receptor antagonism in addition to major histocompatibility complex blocking. Proc Natl Acad Sci USA 96:634-639.
17. Arnon R, Aharoni R (2004) Mechanism of action of glatiramer acetate in multiple sclerosis and its potential for the development of new applications. Proc Natl Acad Sci USA 101 Suppl 2:14593-14598.
18. Teitelbaum D, Aharoni R, Sela M, Arnon R (1991) Cross-reactions and specificities of monoclonal antibodies against myelin basic protein and against the synthetic copolymer 1. Proc Natl Acad Sci USA 88:9528-9532.
19. Webb C, Teitelbaum D, Arnon R, Sela M (1973) In vivo and in vitro immunological cross-reactions between basic encephalitogen and synthetic basic polypeptides capable of suppressing experimental allergic encephalomyelitis. Eur J Immunol 3:279-286.
20. Duda et al. (2000) Human and murine CD4 T cell reactivity to a complex antigen: recognition of the synthetic random polypeptide glatiramer acetate. J Immunol 165:7300-7307.
21. Duda P W, et al. (2000) Glatiramer acetate (Copaxone) induces degenerate, Th2-polarized immune responses in patients with multiple sclerosis. J Clin Invest 105:967-976.
22. Brenner T, et al. (2001) Humoral and cellular immune responses to Copolymer 1 in multiple sclerosis patients treated with Copaxone. J Neuroimmunol 115:152-160.
23. Aharoni R, Teitelbaum D, Sela M, Arnon R (1997) Copolymer 1 induces T cells of the T helper type 2 that crossreact with myelin basic protein and suppress experimental autoimmune encephalomyelitis. Proc Natl Acad Sci USA 94:10821-10826.
24. Wiesemann E, et al. (2003) Correlation of serum IL-13 and IL-5 levels with clinical response to Glatiramer acetate in patients with multiple sclerosis. Clin Exp Immunol 133:454-460.
25. Aharoni R, Teitelbaum D, Sela M, Arnon R (1998) Bystander suppression of experimental autoimmune encephalomyelitis by T cell lines and clones of the Th2 type induced by copolymer 1. J Neuroimmunol 91:135-146.
26. Aharoni R, et al. (2003) Glatiramer acetate-specific T cells in the brain express T helper ⅔ cytokines and brain-derived neurotrophic factor in situ. Proc Natl Acad Sci USA 100:14157-14162.
27. Miller A, et al. (1998) Treatment of multiple sclerosis with copolymer-1 (Copaxone): implicating mechanisms of Th1 to Th2/Th3 immune-deviation. J Neuroimmunol 92:113-121.
28. Neuhaus O, et al. (2000) Multiple Sclerosis: Comparison of copolymer-1-reactive T cell Lines from treated and untreated subjects reveals cytokine shift From T helper 1 to T helper 2 cells. Proceedings of the National Academy of Sciences 37:7452-7457.
29. Venken K, et al. (2008) Natural naive CD4+CD25+CD1271ow regulatory T cell (Treg) development and function are disturbed in multiple sclerosis patients: recovery of memory Treg homeostasis during disease progression. J Immunol 180:6411-6420.
30. Haas J, et al. (2009) Glatiramer acetate improves regulatory T-cell function by expansion of naive CD4(+)CD25(+)FOXP3(+)CD31(+) T-cells in patients with multiple sclerosis. J Neuroimmunol 216:113-117.
31. Hong J, et al. (2005) Induction of CD4+CD25+ regulatory T cells by copolymer-I through activation of transcription factor Foxp3. Proc Natl Acad Sci USA 102:6449-6454.
32. Weber M S, et al. (2007) Type II monocytes modulate T cell-mediated central nervous system autoimmune disease. Nat Med 13:935-943.

What is claimed is:

1. A process for characterizing a glatiramer acetate related drug substance (GARDS) or a glatiramer acetate related drug product (GARDP) comprising separating a batch of a GARDS or GARDP according to hydrophobicity and determining the molar mass of the separated material, thereby characterizing the GARDS or GARDP by molar mass as a function of hydrophobicity.

2. The process of claim 1 further comprising a step of producing a profile of the molar mass of the GARDS or GARDP.

3. The process of claim 1 or claim 2, wherein separating is performed by eluting the batch of the GARDS or GARDP using chromatography with a mobile phase.

4. The process of claim 3, wherein the chromatography is reversed-phase chromatography.

5. The process of claim 4, wherein the reversed-phase chromatography is reversed-phase high-performance liquid chromatography.

6. The process of any one of claims 3-5, wherein the chromatography is performed with a gradient elution of the mobile phase.

7. The process of claim 6, wherein the gradient, elution is achieved by using organic solvent up to 50% by volume of the mobile phase.

8. The process of claim 7, wherein the organic solvent is 0.1% trifluoroacetic acid in acetonitrile.

9. The process of any one of claims 1-8, wherein the batch of the GARDS or GARDP is separated into a continuous stream having varying hydrophobicity and the molar mass of at least a portion of the continuous stream is determined.

10. The process of any one of claims 1-8, wherein the batch of the GARDS or GARDP is separated into separate fractions having varying hydrophobicity and the molar mass of a separated fraction is determined.

11. The process of any one of claims 1-10, wherein the molar mass is determined using a Multi Angle Laser Light Scattering (MALLS) instrument.

12. The process of any one of claims 2-11, wherein the profile is a profile of molar mass as a function of hydrophobicity.

13. A process for discriminating between two or more GARDSs or GARDPs comprising:
(I) characterizing two or more GARDSs or GARDPs according to the process of any one of claims 1-12 to obtain a profile of molar mass as a function of hydrophobicity for each of the two or more GARDS or GARDP; and
(II) comparing each of the profiles obtained in step (I) to each other,
thereby discriminating between the GARDSs or GARDPs.

14. The process of claim 13, wherein the characterization is by chromatography, further comprising the step of identifying the GARDSs or GARDPs as not substantially equivalent if:
(a) the peak molar mass of the GARDSs or GARDPs according to the profiles are different; or
(b) the retention time at the peak of the profiles of the GARDSs or GARDPs are different.

15. A process for producing a drug product comprising a GARDS, which involves an array of testing, comprising including in the array of testing:
(I) characterizing a GARDS according to the process of any one of claims 1-12 to obtain a profile of molar mass as a function of hydrophobicity for the GARDS;
(II) characterizing glatiramer acetate drug substance (GADS) according to the same conditions used in step (I) to obtain a profile of molar mass as a function of hydrophobicity for GADS; and
(III) including the GARDS in the production of the drug product if the profile obtained in step (I) is substantially equivalent to the profile obtained in step (II).

16. A process for producing a drug product comprising a GARDS, which involves an array of testing, comprising including in the array of testing:
(I) characterizing a GARDS according to the process of any one of claims 1-12 to obtain a profile of molar mass as a function of hydrophobicity for the GARDS; and
(II) including the GARDS in the production of the drug product if the profile obtained in step (I) is substantially equivalent to the profile representing glatiramer acetate drug substance (GADS) when characterized under the same conditions as the conditions used in step (I).

17. A process for producing a drug product comprising a GARDS, which involves an array of testing, comprising including in the array of testing:
(a) characterizing a GARDS according to the process of any one of claims 1-12 to obtain a profile of molar mass as a function of hydrophobicity for the GARDS; and
(b) including the GARDS in the production of the drug product if the profile has a single peak and the peak molar mass of the GARDS according to the profile is in the range of 8,000-10,000 g/mol.

18. The process of 17, wherein the characterization is by chromatography, further comprising:
(I) characterizing glatiramer acetate drug substance (GADS) according to the same conditions used in step (a) to obtain a profile of molar mass as a function of hydrophobicity for GADS; and
(II) including the GARDS in the production of the drug product if the chromatography retention time at the peak molar mass of the GARDS is substantially equivalent to the chromatography retention time at the peak molar mass of the GADS.

19. A process for releasing a drug product comprising a GARDS, which involves an array of testing, comprising including in the array of testing:
(I) characterizing a GARDS according to the process of any one of claims 1-12 to obtain a profile of molar mass as a function of hydrophobicity for the GARDS;
(II) characterizing glatiramer acetate drug substance (GADS) according to the same conditions used in step (I) to obtain a profile of molar mass as a function of hydrophobicity for GADS; and
(III) releasing the drug product if the profile obtained in step (I) is substantially equivalent to the profile obtained in step (II).

20. A process for releasing a drug product comprising a GARDS, which involves an array of testing, comprising including in the array of testing:
(I) characterizing a GARDS according to the process of any one of claims 1-12 to obtain a profile of molar mass as a function of hydrophobicity for the GARDS; and
(II) releasing the drug product if the profile obtained in step (I) is substantially equivalent to the profile representing glatiramer acetate drug substance (GADS) when characterized under the same conditions as the conditions used in step (I).

21. A process for releasing a drug product comprising a GARDS, which involves an array of testing, comprising including in the array of testing:
(a) characterizing a GARDS according to the process of any one of claims 1-12 to obtain a profile of molar mass as a function, of hydrophobicity for the GARDS; and
(b) releasing the drug product if the profile has a single peak and the peak molar mass of the GARDS according to the profile is in the range of 8,000-10,000 g/mol.

22. The process of 21, wherein the characterization is by chromatography, further comprising:
(I) characterizing glatiramer acetate drug substance (GADS) according to the same conditions used in step (a) to obtain a profile of molar mass as a function of hydrophobicity for GADS; and
(II) releasing the drug product if the chromatography retention time at the peak molar mass of the GARDS is substantially equivalent to the chromatography retention time at the peak molar mass of the GADS.

23. A process for identifying GARDS or GARDP that has suboptimal activity comprising:
(I) characterizing a GARDS according to the process of any one of claims 1-12 to obtain a profile of molar mass as a function of hydrophobicity for the GARDS;
(II) characterizing glatiramer acetate drug substance (GADS) according to the same conditions used in step (I) to obtain a profile of molar mass as a function of hydrophobicity for GADS; and
(III) identifying the GARDS or GARDP as having a suboptimal activity if the profile obtained in step (I) is not substantially equivalent to the profile obtained in step (II).

24. A process for identifying GARDS or GARDP that has suboptimal activity comprising:
(I) characterizing a GARDS according to the process of any one of claims 1-12 to obtain a profile of molar mass as a function of hydrophobicity for the GARDS;
(II) identifying the GARDS or GARDP as having a suboptimal activity if the profile obtained in step (I) is not substantially equivalent to the profile representing glatiramer acetate drug substance (GADS) when characterized under the same conditions as the conditions used in step (I).

25. A process for identifying GARDS or GARDP that has suboptimal activity comprising:
(a) characterizing a GARDS according to the process of any one of claims 1-12 to obtain a profile of molar mass as a function of hydrophobicity for the GARDS; and
(b) identifying the GARDS or GARDP as having a suboptimal activity if the profile has more than one peak or the peak molar mass of the GARDS according to the profile is not in the range of 8,000-10,000 g/mol.

26. The process of 25, wherein the characterization is by chromatography, further comprising:
(I) characterizing glatiramer acetate drug substance (GADS) according to the same conditions used in step (a) to obtain a profile of molar mass as a function of hydrophobicity for GADS; and
(II) identifying the GARDS or GARDP as having a suboptimal activity if the chromatography retention time at the peak molar mass of the GARDS is not substantially equivalent to the chromatography retention time at the peak molar mass of the GADS.
